(19) United States
(12) Patent Application Publication
(10) Pub. No.: US 2024/0342374 A1
(43) Pub. Date: Oct. 17, 2024

(54) Syringe Adapter with Needle Hub

(71) Applicant: Becton, Dickinson and Copany, Franklin Lakes, NJ (US)

(72) Inventors: Tianqi Hang, Cliffside Park, NJ (US); Nicole Jean DiStefano, Dumont, NJ (US); Balaji Sadasivam, Mahwah, NJ (US); Rafael Gonzalez Macias, San Diego, CA (US); Noel Antonio Medrano, Cary, NC (US); Shivani Rajesh Patel, Jersey City, NJ (US)

(21) Appl. No.: 18/682,140

(22) PCT Filed: Aug. 12, 2022
(86) PCT No.: PCT/US22/40143

§ 371 Date: Feb. 8, 2024

Related U.S. Application Data
(60) Provisional application No. 63/232,454, filed on Aug. 12, 2021.

(51) Int. Cl. A61M 5/178; A61M 5/34; A61M 39/10

(52) U.S. Cl. CPC A61M 5/1782; A61M 5/343; A61M 2039/1066

(57) ABSTRACT

A syringe adapter includes an outer housing having a sidewall defining an interior space, a membrane housing positioned within the interior space of the outer housing and moveable from a first position within the outer housing to a second position within the outer housing spaced from the first position, with the membrane housing receiving a membrane, a needle hub connected to the outer housing and configured to be connected to a syringe, with the needle hub rotatable relative to the outer housing, and a needle connected to the needle hub. The needle having a first end and a second end positioned opposite the first end, with the second end of the needle received within the membrane when membrane housing is in the first position. The needle is rotatable relative to the needle hub.

### CROSS-REFERENCE TO RELATED APPLICATION

The present application claims priority to U.S. Provisional Application No. 63/232,454, entitled “Syringe Adapter with Needle Hub”, filed Aug. 12, 2021, the entire disclosure of which is hereby incorporated by reference in its' entirety.

### BACKGROUND OF THE INVENTION

### Field of the Invention

The present invention relates to a syringe adapter with a needle hub.

### Description of Related Art

Health care providers reconstituting, transporting, and administering hazardous drugs, such as chemotherapeutic medication, can put health care providers at risk of exposure to these medications and present a hazard in the health care environment. Unintentional chemotherapy exposure can affect the nervous system, impair the reproductive system, and bring an increased risk of developing blood cancers in the future. Some drugs must be dissolved or diluted before they are administered, which involves transferring a solvent from one container to a sealed vial containing the drug in powder or liquid form. Drugs may be inadvertently released into the atmosphere in gas form or by way of aerosolization, during the withdrawal of the medication from the vial if any pressure differential between the interior of the vial and the surrounding atmosphere exists. In order to reduce the risk of health care providers being exposed to toxic drugs, the transfer of these drugs is accomplished utilizing a closed system transfer device or system.

Syringe adapters are utilized with a syringe to transfer medication from a container, such as a vial, to the syringe or to transfer a diluent from the syringe to the vial to reconstitute a medication. Syringe adapters are also utilized to transfer medication from the syringe to a patient infusion line or to an infusion container, such as an IV bag, via an IV bag spike. Syringe adapters include a membrane received by a membrane housing, with the membrane of the syringe adapter configured to engage a corresponding membrane of a mating component, such as a patient connector, vial adapter, or IV bag spike, to ensure the closed transfer of fluid between the components such that the medication is not exposed to the ambient environment.

### SUMMARY OF THE INVENTION

In one aspect or embodiment, a syringe adapter includes an outer housing having a first end and a second end positioned opposite the first end with a longitudinal axis extending from the first end of the outer housing to the second end of the outer housing, with the outer housing including a sidewall defining an interior space, a membrane housing positioned within the interior space of the outer housing and moveable from a first position within the outer housing to a second position within the outer housing spaced from the first position, with the membrane housing receiving a membrane, a needle hub connected to the outer housing and configured to be connected to a syringe, with the needle hub rotatable relative to the outer housing, and a needle connected to the needle hub, with the needle having a first end and a second end positioned opposite the first end. The second end of the needle is received within the membrane when membrane housing is in the first position. The needle is rotatable relative to the needle hub.

The needle may include a frustoconical surface engaged with a corresponding frustoconical surface of the needle hub. The needle hub may include a central opening, with at least a portion of the needle received within the central opening, and with the needle hub including a protrusion extending radially inward into the central opening and engaged with the first end of the needle.

The needle may be fixed relative to the outer housing. The outer housing may include a needle passageway at the first end of the outer housing, with the needle passageway receiving a portion of the needle.

The needle may include a spherical surface engaged with a corresponding spherical surface of the needle hub.

The syringe adapter may include a seal positioned between the needle and the needle hub. The seal may be an O-ring. The syringe adapter may include a spring received within the outer housing and bias the membrane housing toward the first position.

In a further aspect or embodiment, a syringe adapter includes an outer housing having a first end and a second end positioned opposite the first end with a longitudinal axis extending from the first end of the outer housing to the second end of the outer housing, with the outer housing including a sidewall defining an interior space, a membrane housing positioned within the interior space of the outer housing and moveable from a first position within the outer housing to a second position within the outer housing spaced from the first position, with the membrane housing receiving a membrane, a needle hub connected to the outer housing and configured to be connected to a syringe, with the needle hub rotatable relative to the outer housing and including an extension engaged with the membrane housing. Rotation of the needle hub is configured to rotate the membrane housing via the extension. The syringe adapter further includes a needle connected to the needle hub, with the needle having a first end and a second end positioned opposite the first end. The second end of the needle is received within the membrane when membrane housing is in the first position.

The extension may be received by a recess defined by the membrane housing, with the extension configured to slide within the recess when the membrane housing moves from the first position to the second position. The extension may be configured to bias the membrane housing toward the first position. The extension may be arcuate. The extension may be three equally-spaced extensions.

The membrane housing may include a needle guide configured to engage the needle as the membrane housing moves from the first position to the second position. The needle guide may include a frustoconical surface. The needle guide may extend at least 25 percent of a length of the membrane housing.

A piercing tip of the needle may be positioned within the membrane when the membrane housing is in the first position, with the piercing tip of the needle positioned outside of the membrane when the membrane housing is in the second position. The membrane housing may include a collet configured to receive a connection interface of a mating connector. The extension may be formed integrally with the needle hub.

### BRIEF DESCRIPTION OF THE DRAWINGS

FIG. 1 is a front view of a conventional syringe adapter.

FIG. 2 is a cross-sectional view of the syringe adapter of FIG. 1.

FIG. 3 is a partial cross-sectional view of a syringe adapter according to one aspect or embodiment of the present application.

FIG. 4 is a partial cross-sectional view of the syringe adapter of FIG. 3.

FIG. 5 is a partial cross-sectional view of a syringe adapter according to a further aspect or embodiment of the present application.

FIG. 6 is a cross-sectional view of a needle hub for a syringe adapter according to one aspect or embodiment of the present application.

FIG. 7 is an enlarged view of the needle hub of FIG. 6.

FIG. 8 is a perspective view of a needle hub for a syringe adapter according to a further aspect or embodiment of the present application.

FIG. 9 is a cross-sectional view of the needle hub of FIG. 8.

FIG. 10 is a bottom view of the needle hub of FIG. 8.

FIG. 11 is a perspective view of the needle hub of FIG. 8, showing the needle hub in connection with a seal assembly.

FIG. 12 is a cross-sectional view of a syringe adapter with the needle hub of FIG. 8 according to one aspect or embodiment of the present application.

FIG. 13 is a perspective view of a seal assembly according to one aspect or embodiment of the present application.

FIG. 14 is a partial cross-sectional view of the seal assembly of FIG. 13, with a membrane omitted for clarity.

FIG. 15 is a partial cross-sectional view of the seal assembly of FIG. 13, showing a needle positioned within the seal assembly.

### DETAILED DESCRIPTION

For purposes of the description hereinafter, the terms such as “end”, “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting. Further, it is to be understood that the invention may assume various alternative variations and step sequences, except where expressly specified to the contrary.

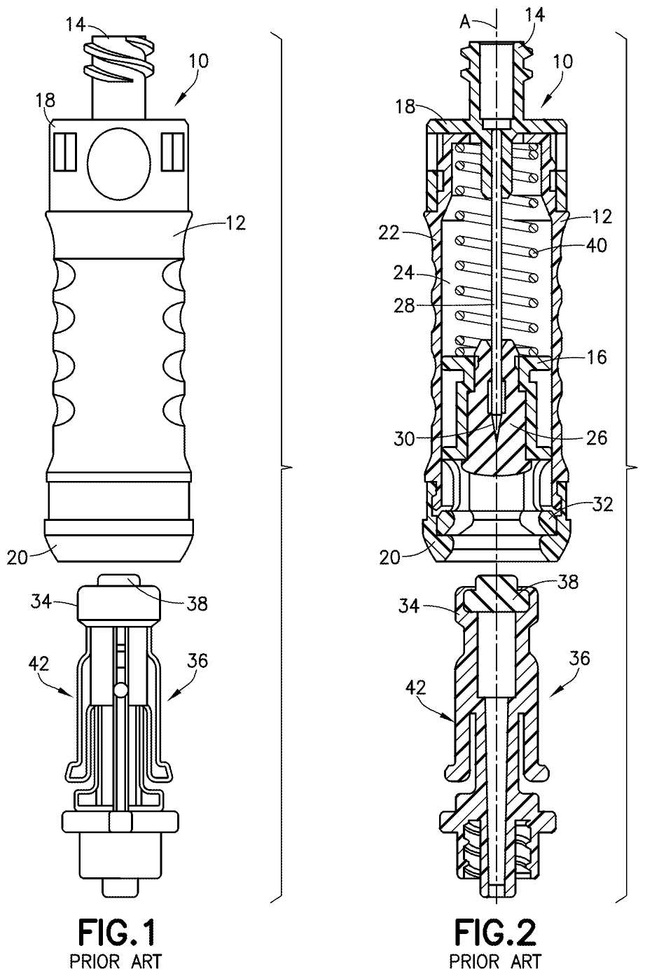

Referring to FIGS. 1 and 2, a conventional syringe adapter 10 is shown. The syringe adapter 10 is one component of a system for the closed transfer of fluids. In particular, the syringe adapter 10 is configured to connect to a syringe (not shown) and to another medical device or fluid container. The medical device can be, for example, a patient line, vial adapter, fluid container, or infusion adapter. The container can be a medical vial, syringe barrel, IV bag, or similar container for holding a fluid to be administered to a patient. The syringe adapter 10 can be used to facilitate the closed transfer of fluids between the syringe and medical device or fluid container. The syringe adapter 10 is shown and described in U.S. Patent Application Publication No. 2018/0200147, which is hereby incorporated by reference in its entirety.

Referring again to FIGS. 1 and 2, the syringe adapter 10 includes an outer housing 12, a needle hub 14 having a syringe connection, and a membrane housing 16. The outer housing 12 has a first end 18 and a second end 20 positioned opposite the first end 18 with a longitudinal axis A extending from the first end 18 of the outer housing 12 to the second end 20 of the outer housing 12. The outer housing 12 includes a sidewall 22 defining an interior space 24. The needle hub 14 is configured to be connected to a syringe. The needle hub 14 may include a luer connector, although other suitable connection arrangements may be utilized. The membrane housing 16 is positioned within the interior space 24 of the outer housing 12 and is moveable from a first position within the outer housing 12 to a second position within the outer housing 12 that is spaced from the first position. The membrane housing 12 receives a membrane 26. The syringe adapter 10 further includes a needle 28 received within the interior space 24 of the outer housing 12. A piercing tip 30 of the needle 28 is positioned within the membrane 26 when the membrane housing 16 is in the first position and the piercing tip 30 of the needle 28 is positioned outside of the membrane 26 when the membrane housing 16 is in the second position. The needle 28 is secured to the needle hub 14 and rotates with the needle hub 14. In particular, the needle hub 14 is rotatable relative to the outer housing 12, which inhibits accidental disconnection of a syringe from the needle hub 14 by requiring a healthcare worker to grasp the needle hub 14 to connect and disconnect a syringe from the needle hub 14. The membrane housing 16 includes a collet 32 configured to receive a connection interface 34 of a mating connector 36, such as a patient connecter as shown in FIGS. 1 and 2. The mating connector 36 includes a membrane 38. The mating connector 36 may be other components of a closed system transfer device or system, such as a vial adapter or IV bag spike. The syringe adapter 10 also includes a spring 40 received within the interior space 24 of the outer housing 12. The spring 40 biases the membrane housing 16 toward the first position.

When the mating connector 36 is inserted into the outer housing 16 of the syringe adapter 10, the collet 32 of the membrane housing 16 receives the connection interface 34 of the mating connector 36 and moves the membrane housing 16 within the outer housing 12 toward the first end of the outer housing 12. As the membrane housing 16 moves, the collet 32 secures the membrane housing 16 to the mating connector 36 to ensure engagement between the membrane 38 of the mating connector 36 and the membrane 26 of the membrane housing 16. As the membrane housing 16 is moved from the first position to the second position, the piercing tip 30 of the needle 28 pierces the membrane 26 of the membrane housing 16 and the membrane 38 of the mating connector 36 such that the piercing tip 30 is received within the mating connector 36 thereby placing the needle 28 in fluid communication with the mating connector 36. The needle hub 14 is in fluid communication with the needle 28 to allow a syringe (not shown) connected to the needle hub 14 to be in fluid communication with the mating connector 36 when the syringe adapter 10 is connected to the mating connector 36. Moving the membrane housing 16 from the second position to the first position, which is aided by the spring 40, releases the mating connector 36 from the collet 32 to separate the mating connector 36 from the syringe adapter 10. The membrane housing 16 is secured in the second position by a locking arrangement 42 of the mating connector 36 that engages a portion of the outer housing 12 to hold the membrane housing 16 in the second position. Pulling the mating connector 36 away from the syringe adapter 10 releases the locking arrangement 42 to allow the membrane housing 16 to move back to the first position from the second position.

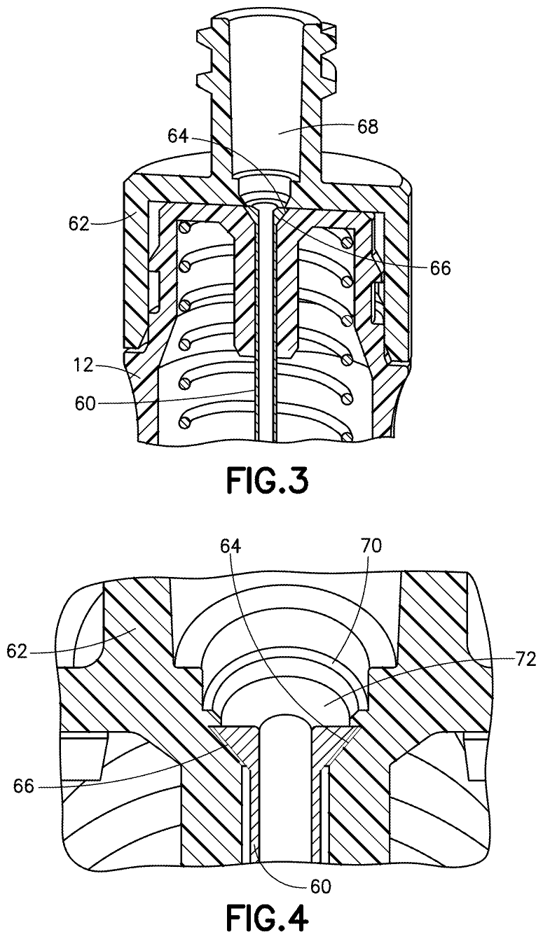

Referring to FIGS. 3 and 4, in accordance with one aspect or embodiment, the syringe adapter 10 includes a needle 60 that is rotatable relative to a needle hub 62. Rather than providing the needle hub 14 that is fixed relative to the needle 28, as shown in FIGS. 1 and 2, the needle 60 of FIGS. 3 and 4 is free to rotate relative to the needle hub 62 such that rotation of the needle hub 62 relative to the outer housing 12 is not translated to the needle 60 thereby minimizing variation in the location of where the needle 60 penetrates the membrane 26. Minimizing variations in the location of where the needle 60 penetrates the membrane 26 prevents fragmentation of the membrane 26 during use of the syringe adapter 10. The needle 60 is connected to the needle hub 62 to prevent axial movement of the needle 60 relative to the needle hub 62. The needle 60 includes a frustoconical surface 64 engaged with a corresponding frustoconical surface 66 of the needle hub 62. The needle hub 62 includes a central opening 68, with at least a portion of the needle 60 received within the central opening 68. The needle hub 62 includes a protrusion 70 extending radially inward into the central opening 68, with the protrusion 70 engaged with a first end 72 of the needle 60. The engagement of the frustoconical surfaces 64,66 of the needle 60 and the needle hub 62 prevents axial movement of the needle 60 relative to the needle hub 62 in a first direction while the protrusion 70 prevents axial movement of the needle 60 relative to the needle hub 62 in a second direction, which is opposite to the first direction. The frustoconical surfaces 64,66 of the needle 60 and the needle hub 62 provides a mating surface to allow smooth rotational movement of the needle 60 relative to the needle hub 62.

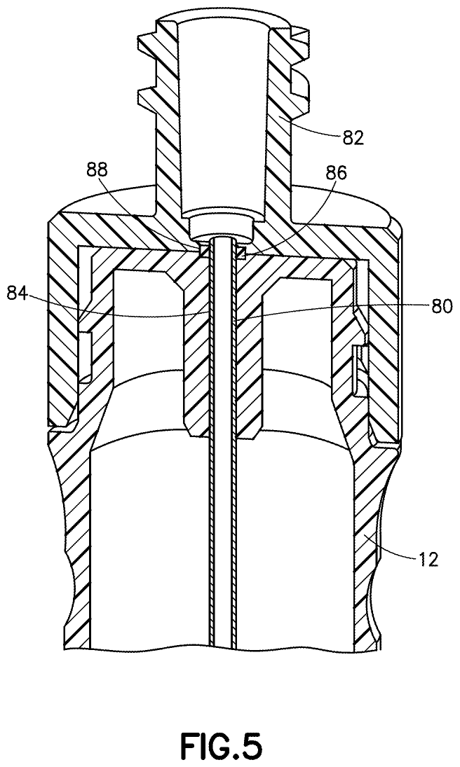

Referring to FIG. 5, in accordance with a further aspect or embodiment, the syringe adapter 10 includes a needle 80 that is rotatable relative to a needle hub 82 similar to the needle 60 and the needle hub 62 of FIGS. 3 and 4. The needle 80 is fixed relative to the outer housing 12. More specifically, in one aspect or embodiment, the outer housing 12 includes a needle passageway 84 at the first end 18 of the outer housing 12, with the needle passageway 84 receiving a portion of the needle 80. A seal 86 is positioned between the needle 80 and the needle hub 82. In one aspect or embodiment, the seal 86 is an O-ring, although other suitable seals may be utilized. The seal 86 may be received within a recess 88 defined by at least one of the needle hub 82 and the outer housing 12. With the needle 80 fixed relative to the outer housing 12, rotation of the needle hub 82 is not translated to the needle 80 in a similar manner as the needle hub 62 of FIGS. 3 and 4. The seal 86 allows the needle hub 82 to rotate relative to the needle 80 while ensuring a liquid tight seal between the needle hub 82 and the needle 80.

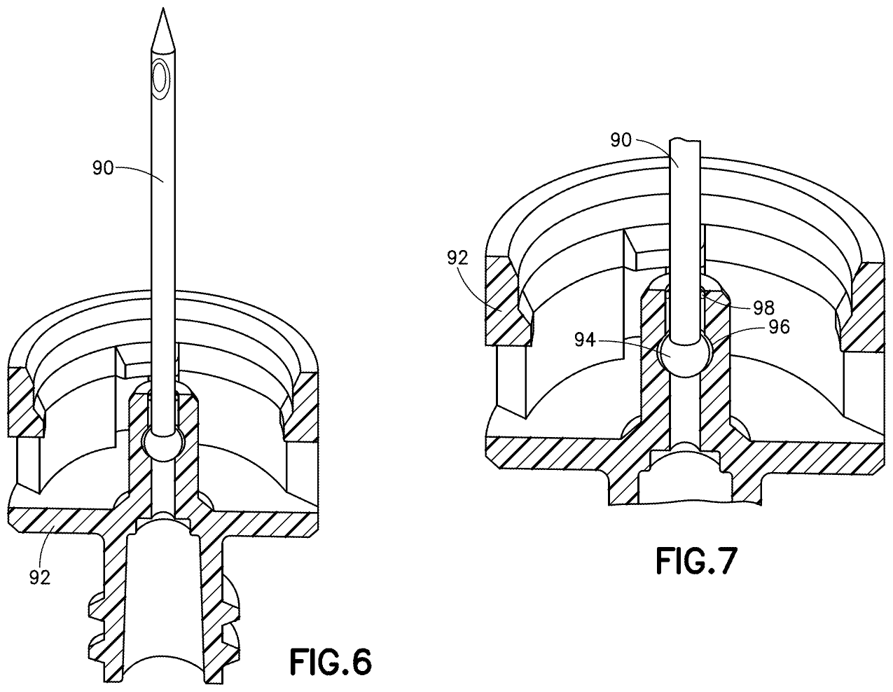

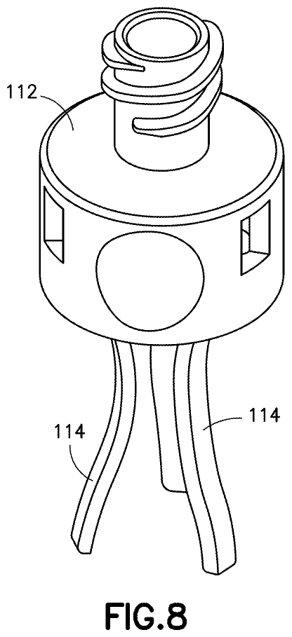

Referring to FIGS. 6 and 7, in accordance with a further aspect or embodiment, the syringe adapter 10 includes a needle 90 that is rotatable relative to a needle hub 92 similar to the needle 60,80 and the needle hub 62,82 arrangements of FIGS. 3-5. The needle 90 of FIGS. 6 and 7 includes a spherical surface 94 engaged with a corresponding spherical surface 96 of the needle hub 92. The needle hub 92 and the needle 90 of FIGS. 6 and 7 also includes a seal 98 positioned between the needle hub 92 and the needle 90. In one aspect or embodiment, the seal 98 is an O-ring, although other suitable seals may be utilized. The seal 98 may be received within a recess defined by at least one of the needle hub 92 and the outer housing 12. Rotation of the needle hub 92 is not translated to the needle 90 in a similar manner as the needle hubs 62,82 of FIGS. 3-5. The seal 98 allows the needle hub 92 to rotate relative to the needle 90 while ensuring a liquid tight seal between the needle hub 92 and the needle 90. The spherical surfaces 94,96 of the needle 90 and the needle hub 92 prevents relative axial movement between the needle 90 and the needle hub 92 while allowing the needle hub 92 to rotate relative to the needle 90.

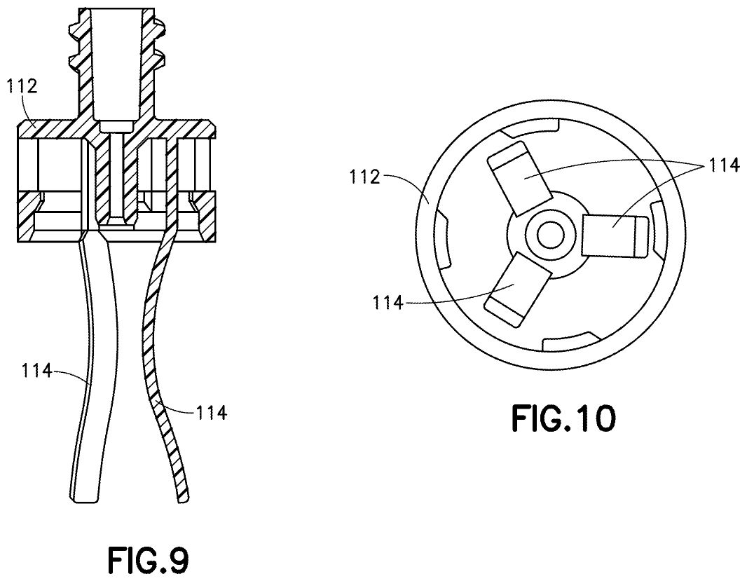

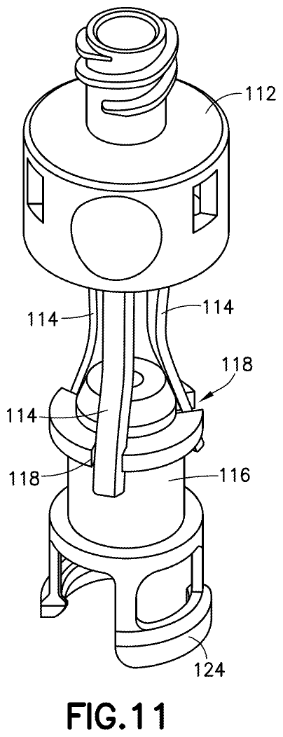

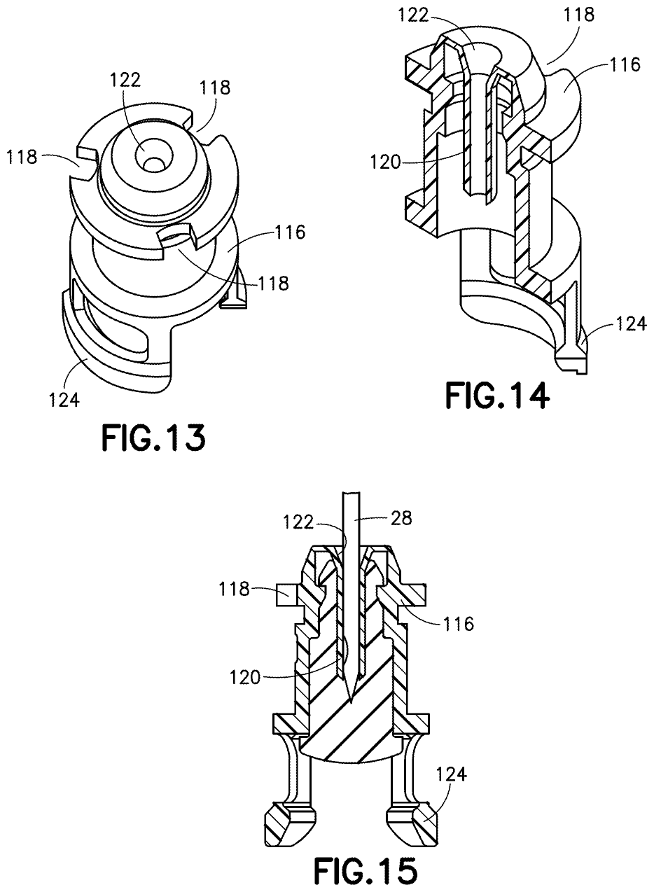

Referring to FIGS. 8-15, in accordance with a further aspect or embodiment, a syringe adapter 110 is shown that includes similar features and functions similarly to the syringe adapter 10 shown in FIGS. 1 and 2, except for several features as discussed below. The syringe adapter 110 of FIGS. 8-15 includes a needle hub 112 connected to the outer housing 12 and includes an extension 114 engaged with a membrane housing 116, with rotation of the needle hub 112 configured to rotate the membrane housing 116 via the extension 114. The extension 114 of the syringe adapter 110, as discussed below, is configured to rotate the membrane housing 116, the membrane 26, and the needle hub 112 as a unit, which minimizes fragmentation of the membrane 26 caused by puncturing of the membrane 26 at multiple locations. The extension 114 is also configured to center the membrane housing 116 and the membrane 26 relative to the needle 28 and also act as a spring to bias membrane housing 116 within the outer housing 12 as discussed above. In particular, the extension 114 is configured to bias the membrane housing 116 toward the first position.

Referring to FIGS. 8-12, the needle hub 112 includes three-equally spaced extensions 114, although one or more extensions 114 may be provided. Each extension 114 is arcuate, although other suitable shapes and configurations may be utilized. A concave surface of each extension 114 faces radially outward toward the outer housing 12. The extension(s) 114 is formed integrally with the needle hub 112, although other suitable arrangements may be utilized.

Referring to FIGS. 8-15, the extension(s) 114 of the needle hub 112 is received by a recess(es) 118 defined by the membrane housing 116, with the extension(s) 114 configured to slide within the recess(es) 118 when the membrane housing 116 moves from the first position to the second position. As the membrane housing 116 moves from the first position to the second position during use of the syringe adapter 110, the extension(s) 114 slides within the recess(es) 118, with the membrane housing 116 pushing against the extension(s) 114 and flattening the extension(s) 114 thereby providing a biasing force to move the membrane housing 116 back to the first position. Due to the resilient properties of the extension(s) 114, the extension(s) 114 wants to return to the original shape of the extension(s) 114 after being flattened, which provides the biasing force. The biasing force provided by the extension(s) 114 removes the need for the spring 40 shown in FIGS. 1 and 2.

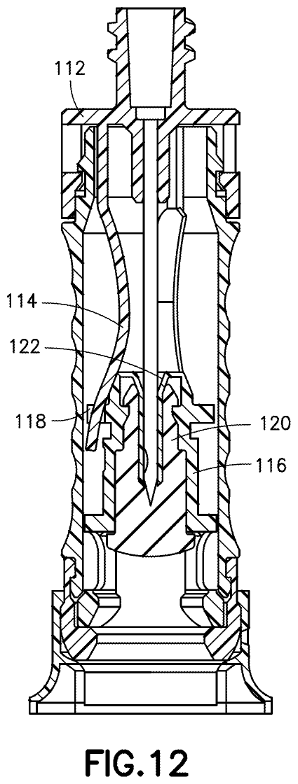

Referring to FIGS. 12, 14, and 15, the membrane housing 116 includes a needle guide 120 configured to engage the needle 28 as the membrane housing 116 moves from the first position to the second position. The needle guide 120 is cylindrical and includes a frustoconical surface 122. The frustoconical surface 122 of the needle guide 120 is configured to guide the initial insertion of the needle 28 into the needle guide 120 during assembly of the syringe adapter 110. The needle guide 120 extends at least 25 percent of a length of the membrane housing 116, although other suitable lengths may be utilized. The needle guide 120 is received within the membrane 26 and is configured to minimize multiple penetration sites from the needle 28 into the membrane 26. The piercing tip 30 of the needle 28 is positioned within the membrane 26 when the membrane housing 116 is in the first position. The piercing tip 30 of the needle 28 is positioned outside of the membrane 26 when the membrane housing 116 is in the second position. The membrane housing 116 includes a collet 124 configured to receive the connection interface 34 of the mating connector 36.

Referring again to FIGS. 8-15, during use of the syringe adapter 110, when the needle hub 112 is rotated, the membrane housing 116, the membrane 26, and the needle 28 will also rotate together with the needle hub 112. The rotation of the needle hub 112 is translated to the membrane housing 116 via the extension(s) 114 received within the recess(es) 118 of the membrane housing 116 while still allowing axial movement of the membrane housing 116 within the outer housing 12 and relative to the needle hub 112. Providing equally-spaced extensions 114, such as three equally-spaced extensions 114 as shown, is configured to apply equal force to the membrane housing 116 thereby centering the membrane housing 116 within the outer housing 12 during axial movement of the membrane housing 116.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims. To the extent possible, one or more features of any aspect or embodiment discussed above can be combined with one or more features of any other aspect or embodiment.

1. A syringe adapter comprising:
an outer housing having a first end and a second end positioned opposite the first end with a longitudinal axis extending from the first end of the outer housing to the second end of the outer housing, the outer housing includes a sidewall defining an interior space;
a membrane housing positioned within the interior space of the outer housing and moveable from a first position within the outer housing to a second position within the outer housing spaced from the first position, the membrane housing receiving a membrane;
a needle hub connected to the outer housing and configured to be connected to a syringe, the needle hub is rotatable relative to the outer housing; and
a needle connected to the needle hub, the needle having a first end and a second end positioned opposite the first end, the second end of the needle received within the membrane when membrane housing is in the first position, the needle is rotatable relative to the needle hub.

2. The syringe adapter of claim 1, wherein the needle comprises a frustoconical surface engaged with a corresponding frustoconical surface of the needle hub.

3. The syringe adapter of claim 2, wherein the needle hub comprises a central opening, at least a portion of the needle received within the central opening, and wherein the needle hub comprises a protrusion extending radially inward into the central opening, the protrusion engaged with the first end of the needle.

4. The syringe adapter of claim 1, wherein the needle is fixed relative to the outer housing.

5. The syringe adapter of claim 4, wherein the outer housing comprises a needle passageway at the first end of the outer housing, the needle passageway receiving a portion of the needle.

6. The syringe adapter of claim 1, wherein the needle comprises a spherical surface engaged with a corresponding spherical surface of the needle hub.

7. The syringe adapter of claim 1, further comprising a seal positioned between the needle and the needle hub.

8. The syringe adapter of claim 5, wherein the seal comprises an O-ring.

9. The syringe adapter of claim 1, further comprising a spring received within the outer housing and biasing the membrane housing toward the first position.

10. A syringe adapter comprising:
an outer housing having a first end and a second end positioned opposite the first end with a longitudinal axis extending from the first end of the outer housing to the second end of the outer housing, the outer housing includes a sidewall defining an interior space;
a membrane housing positioned within the interior space of the outer housing and moveable from a first position within the outer housing to a second position within the outer housing spaced from the first position, the membrane housing receiving a membrane;
a needle hub connected to the outer housing and configured to be connected to a syringe, the needle hub is rotatable relative to the outer housing and comprises an extension engaged with the membrane housing, wherein rotation of the needle hub is configured to rotate the membrane housing via the extension; and
a needle connected to the needle hub, the needle having a first end and a second end positioned opposite the first end, the second end of the needle received within the membrane when membrane housing is in the first position.

11. The syringe adapter of claim 10, wherein the extension is received by a recess defined by the membrane housing, the extension configured to slide within the recess when the membrane housing moves from the first position to the second position.

12. The syringe adapter of claim 11, wherein the extension is configured to bias the membrane housing toward the first position.

13. The syringe adapter of claim 10, wherein the extension is arcuate.

14. The syringe adapter of claim 10, wherein the extension comprises three equally-spaced extensions.

15. The syringe adapter of claim 10, wherein the membrane housing comprises a needle guide configured to engage the needle as the membrane housing moves from the first position to the second position.

16. The syringe adapter of claim 15, wherein the needle guide comprises a frustoconical surface.

17. The syringe adapter of claim 16, wherein the needle guide extends at least 25 percent of a length of the membrane housing.

18. The syringe adapter of claim 10, wherein a piercing tip of the needle is positioned within the membrane when the membrane housing is in the first position, and wherein the piercing tip of the needle is positioned outside of the membrane when the membrane housing is in the second position.

19. The syringe adapter of claim 10, wherein the membrane housing comprises a collet configured to receive a connection interface of a mating connector.

20. The syringe adapter of claim 10, wherein the extension is formed integrally with the needle hub.
